(19) United States
(12) Patent Application Publication
(10) Pub. No.: US 2025/0303167 A1
(43) Pub. Date: Oct. 2, 2025

(54) Non-Invasive Intranasal Neuromodulation System

(71) Applicant: Icahn School of Medicine at Mount Sinai, New York, NY (US)

(72) Inventors: Alfred Marc Iloreta, JR., New York, NY (US); Alexis Bruhat, New York, NY (US); Turner Baker, New York, NY (US); Benjamin Rapoport, New York, NY (US); Thomas Oxley, New York, NY (US)

(73) Assignee: Icahn School of Medicine at Mount Sinai, New York, NY (US)

(21) Appl. No.: 18/866,723

(22) PCT Filed: May 19, 2023
(86) PCT No.: PCT/US2023/022847

§ 371 Date: Nov. 18, 2024

Related U.S. Application Data
(60) Provisional application No. 63/343,853, filed on May 19, 2022.

(51) Int. Cl. A61N 1/36; A61N 1/05

(52) U.S. Cl. CPC A61N 1/36103; A61N 1/0558; A61N 1/3615

(57) ABSTRACT

A non-invasive intranasal neuromodulation system is configured to stimulate the sphenopalatine ganglion (SPG) to increase the collateral blood flow and mitigate brain perfusion to treat the symptoms and improve outcomes of acute stroke care, the non-invasive intranasal neuromodulation system comprising a catheter having a first balloon coupled to and surrounding the catheter,′ the first balloon including at least one electrode; and a second balloon coupled to and surrounding the catheter; wherein the first balloon and the second balloon are configured and are spaced apart from one another such that in inflated states of the first balloon and the second balloon, the first balloon is configured to contact or be proximate to the sphenopalatine ganglion to permit stimulation thereof by actuation of the at least one electrode, while the second balloon is configured to hold the non-invasive neuromodulation device in place within the nasopharynx.

### CROSS-REFERENCE TO RELATED APPLICATION

The present application claims priority to and the benefit of U.S. patent application Ser. No. 63/343,853, filed on May 19, 2022, which is hereby incorporated by reference in its entirety.

### TECHNICAL FIELD

The present disclosure is directed to a system and method for treating an acute ischemic stroke (AIS) and more particularly, to a non-invasive intranasal neuromodulation system that is configured to stimulate the sphenopalatine ganglion (SPG) to increase the collateral blood flow and mitigate brain perfusion to treat the symptoms and improve outcomes of acute stroke care. The present system is designed to be deployed in any clinical setting once the patient has been diagnosed with an acute ischemic stroke.

### BACKGROUND

Stroke is the leading cause of disability and the fifth leading cause of death in the United States. Approximately 795,000 people experience a new or recurrent stroke each year. Acute ischemic stroke (AIS) occurs when an obstruction within a blood vessel decreases cerebral blood flow, depriving neurons of oxygen and leading to severe metabolic failure and neural death. The current gold standard of treatment is mechanical thrombectomy, where the blood clot is manually removed with catheter-based devices. Since thrombectomy procedure requires a highly trained neurointerventionalist and multiple imaging tests to conform and assess ischemic stroke, the door-to-treatment times can be extensive and result in reductions of successful functional outcomes following recanalization. This is especially detrimental as prolonged periods of transfer result in less neural tissue that is salvageable by thrombectomy. Another treatment is intravenous tissue plasminogen activator (IVtPA); however, the window of efficacy for such treatments is also very short. There is a need for devices that can be deployed in patient transfer settings that extend the window-of-efficiency for thrombectomy.

### SUMMARY

In accordance with one embodiment, the present disclosure is directed to a non-invasive neuromodulation device that is configured to stimulate the sphenopalatine ganglion for increasing collateral blood flow to the brain to preserve neural tissue following an acute ischemic stroke. The device includes a catheter having a proximal end and an opposing distal end and has an inner lumen formed therein. The device also has a first balloon coupled to and surrounding the catheter. The first balloon includes at least one electrode. A second balloon is coupled to and surrounds the catheter. The first balloon and the second balloon are configured and are spaced apart from one another such that in inflated states of the first balloon and the second balloon, the first balloon is configured to contact or be proximate to the sphenopalatine ganglion to permit stimulation thereof by actuation of the at least one electrode, while the second balloon is configured to hold the non-invasive neuromodulation device in place within the nasopharynx.

A method of stimulating the sphenopalatine ganglion for increasing collateral blood flow to the brain to preserve neural tissue following an acute ischemic stroke is also disclosed and includes the steps of: deploying a non-invasive neuromodulation device intranasally and electrically stimulating the sphenopalatine ganglion with at least one electrode located on a balloon.

More specifically, a method is disclosed for stimulating the sphenopalatine ganglion for increasing collateral blood flow to the brain to preserve neural tissue following an acute ischemic stroke. The method includes the steps of:
deploying a non-invasive neuromodulation device intranasally, the non-invasive neuromodulation device including a catheter, a first balloon and a second balloon, the first balloon being coupled to and surrounding the catheter, the first balloon including at least one electrode, the second balloon coupled to and surrounding the catheter;
positioning the non-invasive neuromodulation device such that the first balloon is located proximate the sphenopalatine ganglion and the second balloon is located within the nasopharynx;
inflating the second balloon to retain the non-invasive neuromodulation device within the nasopharynx;
inflating the first balloon resulting in at least the at least one electrode of the first balloon contacting the sphenopalatine ganglion; and
electrically stimulating the sphenopalatine ganglion using the at least one electrode that is disposed along the first balloon.

### BRIEF DESCRIPTION OF THE DRAWING FIGURES

FIG. 1 is a schematic illustrating a non-invasive intranasal neuromodulation system according to one embodiment;

FIG. 2 is a side elevation view of a balloon catheter that is part of the system of FIG. 1 and shown in a bent state;

FIG. 3 is a side elevation view of the balloon catheter in a linear state;

FIG. 4 is a side elevation view of the balloon catheter showing internal features thereof;

FIG. 5 is another side elevation view of the balloon catheter;

FIG. 6A is a perspective view of a distal section of the balloon catheter showing internal features thereof according to one embodiment;

FIG. 7A is a cross-sectional view of the shaft of the balloon catheter according to one embodiment;

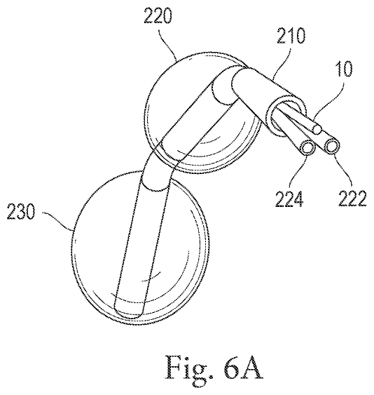

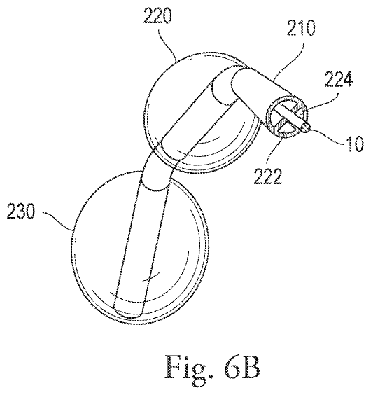

FIG. 6B is a perspective view of a distal section of the balloon catheter showing internal features thereof according to one embodiment;

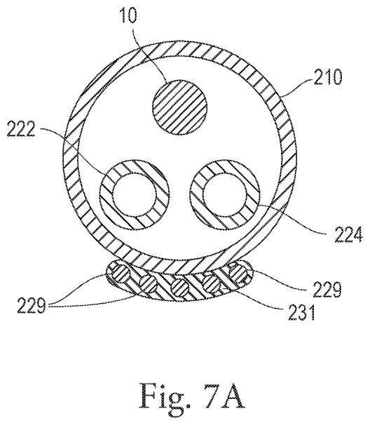

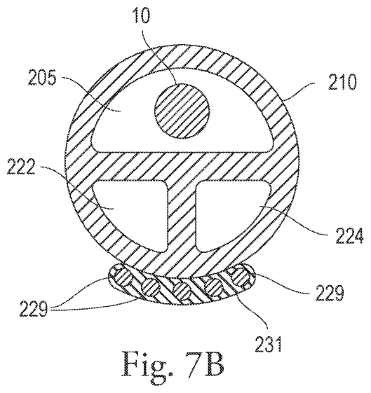

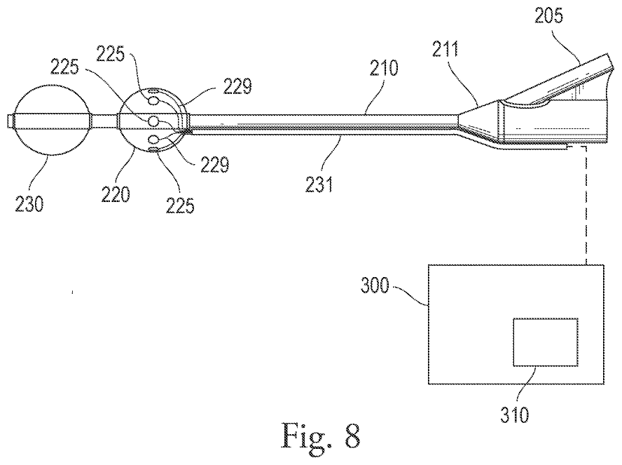

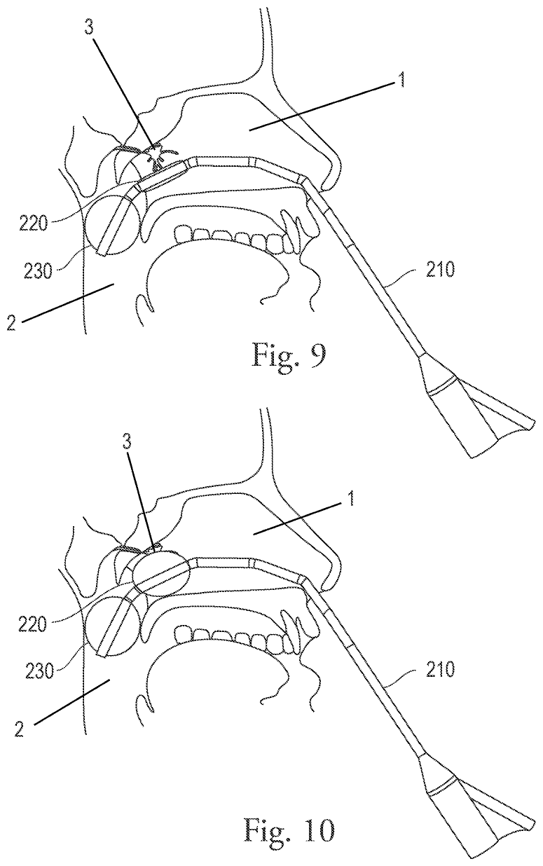

FIG. 7B is a cross-sectional view of the shaft of the balloon catheter according to one embodiment;

FIG. 8 is a schematic view of the non-invasive intranasal neuromodulation system showing one or more electrodes on one balloon;

FIG. 9 shows a first step of inserting the balloon catheter into the nasal cavity and at least partially into the nasopharynx and inflating a distal balloon to anchor the balloon catheter; and

FIG. 10 shows a second step of inflating a proximal balloon so that the one or more electrodes disposed thereon are in contact with the sphenopalatine ganglion.

### DETAILED DESCRIPTION OF CERTAIN EMBODIMENTS

Stimulation of the sphenopalatine ganglion (SPG) (located behind the nose) is proven to increase collateral blood flow in the brain. Dilating target blood vessels with controlled stimulation provides an optimal strategy for extending the window of efficacy for AIS treatments. The present system is configured to stimulate the SPG in a non-invasive way to address this problem. The system is configured to be inserted to be inserted through the nasal opening into the nasal cavity, securing it in place, and then electrically stimulating the SPG. Applying the present system to an AIS stroke patient rapidly benefits the patient in ways beyond those of conventional stroke treatments. Mainly, stimulating the SPG directly with the present system increases the collateral blood flow that alleviates the blood clot due to the stroke, which widens the window for proper stroke treatment. Eventually, the patient has a higher chance of successful surgery and be less at risk of permanent post-stroke damage.

One exemplary non-invasive intranasal neuromodulation system (NINS system or device) for treatment of a stroke patient by alleviating the blood clot due to the stroke is identified at 100. The system 100, as described herein, is designed to be quickly deployed in a number of settings, including patient transfer settings.

The system 100 is formed of two main components, namely, a first component in the form of a balloon catheter 200 that ensures that one or more electrodes 225 (FIG. 8) are placed securely around or in close proximity to the SPG, and a second component in the form of an electrical system 300 for stimulating the SPG using the electrodes 225. Each of these components and the features thereof are described below.

The balloon catheter 200 is designed so that it can safely enter through the nasal opening into the nasal cavity and be positioned at or proximate the SPG. The balloon catheter 200 is thus an elongated structure that is flexible and/or bendable along its length to allow the balloon catheter 200 to travel within the nasal cavity to the target location. As described herein, it will be appreciated that a conventional steering mechanism or the like can be incorporated into the balloon catheter 200 to permit the balloon catheter 200 to navigate anatomical constraints and travel to the target location. In particular, the desired path to the target location is as follows: nasal opening→turbinates→SPG contact→end of the septum→nasopharynx. This path is described in more detail below.

### Balloon Catheter

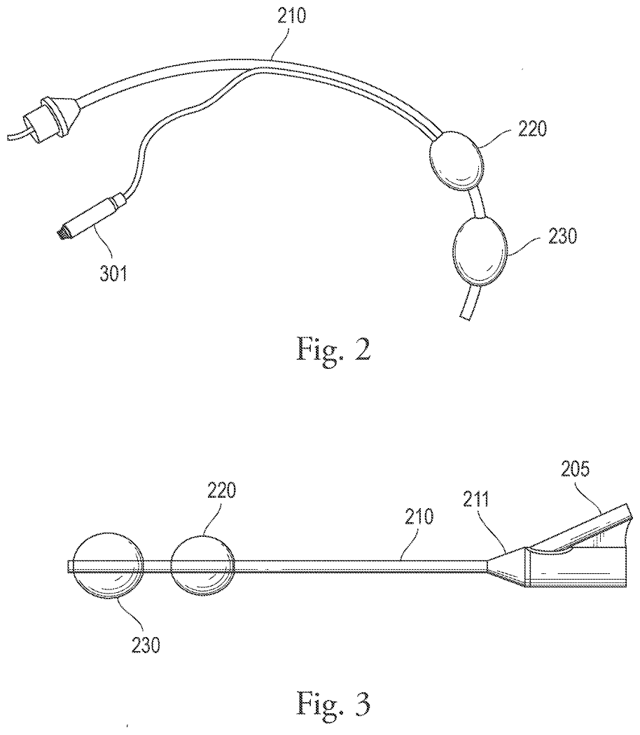

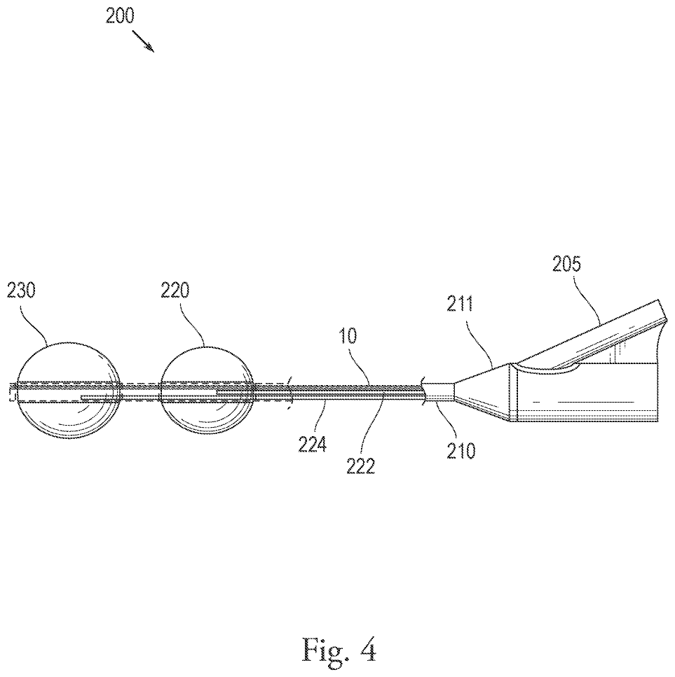

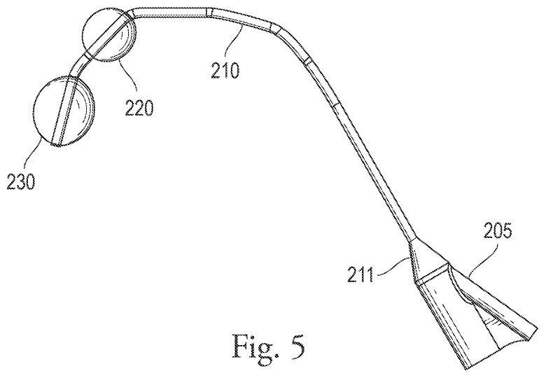

As shown, the balloon catheter 200 is an elongated structure that has a main (bendable) catheter body 210 that has a (first) distal end 202 and a (second) proximal end 204. The proximal end 204 typically has an enlarged area/size relative to a shaft portion of the catheter body 210 that terminates at the distal end 202. This enlarged area can be in the form of a connector or handle 211 that contains various ports and is configured to receive certain elements and be operatively connected to other working elements as described herein. As mentioned herein, the system 100 includes the electrical system 300 for selectively providing energy to electrical components of the balloon catheter 200 and therefore, the electrical system 300 can be connected to or otherwise operatively coupled to the handle 211 as by detachable wiring, etc. In FIG. 2, an electrical connector 301 is shown for electrically connecting the one or more electrodes associated with the balloon catheter 200 to the electrical system 300. The electrical connector 301 can be configured to plug into a port of an electrical stimulation generator (system 300) or the like to electrically connect the one or more electrodes 225 to the electrical stimulation generator.

It will also be appreciated that the electrical stimulation generator can be a self-contained portable unit that can be powered by a battery that is part of the unit. This allows the generator to be carried with the balloon catheter 200 and allows for quick and easy use in various settings such as at a remote location of treatment, in the ambulance, etc.

The balloon catheter 200 includes a pair of inflatable balloons, namely, a first balloon 220 and a second balloon 230 that are spaced apart from one another and are secured to the shaft of the catheter body 210. Each balloon 220, 230 is coupled to the shaft of the catheter body 210 using traditional techniques. The first balloon 220 is located proximal to the second balloon 230 and the second balloon 230 can be located at or proximate to the distal end 202, while the first balloon 220 is spaced from the distal end 202. The first and second balloons 220, 230 operate independent from one another in that the first balloon 220 can be inflated and deflated independent of the second balloon 230 and vice versa. The first and second balloons 220, 230 perform different functions and therefore, they can have different characteristics, such as different sizes, shapes, etc., as described in the below example. For example, the second balloon 230 can have a larger size and larger internal volume relative to the first balloon 220. Both the first balloon 220 and the second balloon 230 can be oval shaped; however, other shapes are equally possible. In addition, the shapes of the two balloons can be the same or different. The spacing between the first and second balloons 220, 230 is selected in view of the functions that each perform to allow each to be placed at the desired anatomical locations once the balloon catheter 200 is inserted into the nasopharynx. In one exemplary embodiment, the spacing is between 2 cm and 20 cm; however, this range is not limiting and the spacing distance can lie outside this range.

It will be understood that different types of inflation media (fluid) can be used to inflate each balloon. For example, the balloon may be filled with saline or with a contrast agent such as Omnipaque that makes the inflated (liquid contrast-filled) balloon visible using fluoroscopy.

As discussed in more detail below, the first balloon 220 can be considered to be a stimulating balloon for placement in the posterior nasal cavity, while the second balloon 230 can be considered to be a locking balloon for placement in the nasopharynx since it is designed and intended to lock and hold the entire balloon catheter 200 in place when inflated.

Since the first and second balloons 220, 230 are independent from one another, each has its own fluid pathway for inflation and deflation. In particular, the balloon catheter 200 has a multi lumen construction and more particularly, the balloon catheter 200 includes a first conduit 222 that is operatively connected to and in fluid communication with the first balloon 220 and similarly, a second conduit 224 is operatively connected to and in fluid communication with the second balloon 230. These two conduits 222, 224 are thus contained within an inner lumen of the catheter body 210 and can be routed in a side-by-side manner.

As shown in FIGS. 6A and 7A, the first conduit 222 can be in the form of a first tube or the like and the second conduit 224 can be in the form of a second tube or the like. The first and second conduits 222, 224 are routed through the balloon catheter body 210 to the locations of the first and second balloon 220, 230. For example, the first conduit 222 can be routed to a location at which the catheter body (shaft) 110 has at least one first hole formed therethrough that opens into the interior of the first balloon 220. In this way, inflation media (fluid) can be delivered through the first conduit 222 to the first balloon 220 for inflation thereof and similarly, when the inflation fluid is removed from the first balloon 220 through the first conduit 222, the balloon deflates. The second conduit 224 can be routed to a location at which the catheter body (shaft) 110 has at least one second hole formed therethrough that opens into the interior of the second balloon 230. In this way, inflation media (fluid) can be delivered through the second conduit 224 to the second balloon 230 for inflation thereof and similarly, when the inflation fluid is removed from the second balloon 230 through the second conduit 224, the second balloon deflates.

FIGS. 6B and 7B illustrate another embodiment in which the first conduit 222 and the second conduit 224 are formed as conduits (passages) formed in the catheter body 210. In other words, the conduits 222, 224 are integrally formed as longitudinal holes formed in the catheter body 210. The guide wire conduit is similarly formed to allow passage of the guide wire 10.

The two conduits 222, 224 are located within the handle and are open at one end of the handle to allow for fluid connection to the two conduits 222, 224. For example, these two conduits 222, 224 are connected to an inflation (fluid) source and typically, a controller that is part of a console or the like controls pumps that both control the delivery of the inflation fluid to the two balloons 220, 230 as well as the removal of the inflation fluid from the two balloons 220, 230. It will be appreciated that a single inflation fluid source can be provided and one or more pumps can be provided to deliver the inflation fluid to the respective balloon. When one pump is used, a manifold can be provided to route the inflation fluid to the proper conduit as by using valves to open and close the conduits 222, 224.

The balloon catheter 200 can also include a guide wire port 205 that is located within the balloon catheter 200 and is open at both ends of the balloon catheter body 210. The guide wire port 205 can thus include a dedicated conduit or lumen through which a guide wire (not shown) is fed. As is known, a guide wire is a thin, semi-flexible, medical wire inserted into a body (here the balloon catheter body 210) to guide the larger instrument (the balloon catheter 200) through the appropriate path to the desired location. The guide wire 10 is thus used to guide the balloon catheter 200 to the target site. The guide wire is generally shown at 10.

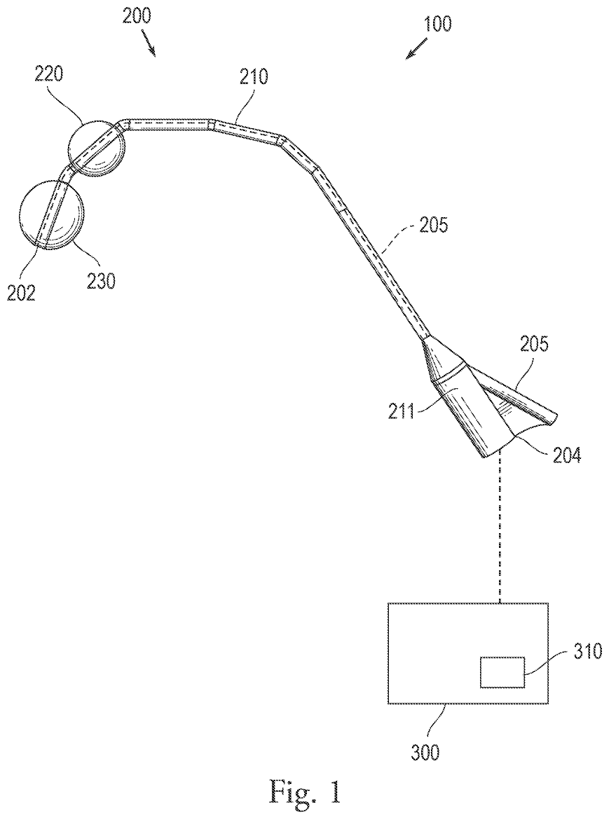

The distal end of the balloon catheter body 210 is thus open to allow the passage of a guide wire that passes through the guide wire port 205 and exits the balloon catheter body 210. As shown in the figures, the guide wire port 205 can be non-linear in nature along its entire length and in particular, the guide wire port 205 can be angled in the handle and then extends linearly or non-linearly along the shaft (as depicted by the dashed lines in FIG. 1). The angled nature of the guide wire port 205 within the handle allows insertion of the guide wire at an off center location, while the fluid connection to the two conduits 222, 224 can be at a more central location within the handle. As shown, the catheter shaft can also be non-linear in nature in that it can include one or more bends formed along its length.

In the deflated state, each of the first balloon 220 and the second balloon 230 is in a collapsed state and seats in its collapsed state against the catheter body 210. This results in the width (diameter) of the catheter body within these two balloon regions being very close to the diameter of the remaining portion of the shaft leading to the compact nature of the balloon catheter 200.

The function performed by the first balloon 220 is that the first balloon 220 is intended to be in contact with or in close proximity to the SPG once the first balloon 220 is fully inflated. As described below, the electrical stimulus is transferred to the SPG through the first balloon 220 and more particularly, by use of the electrodes 225.

Since the first balloon 220 comprises the structure through which electrical stimulus is delivered to the SPG, the first balloon 220 carries one or more electrodes 225 (FIG. 8). The electrodes 225 comprise stimulation electrodes that are capable of providing stimulation. The electrodes 225 thus comprise discrete stimulators located along the balloon's exterior. For sake of simplicity, some figures illustrate the first balloon 220 without electrodes 225; however, it will be understood that the first balloon 220 does include electrodes 225 as described herein and shown in FIG. 8.

The one or more electrodes 225 comprises any number of suitable stimulation electrodes that can be disposed along the exterior surface of the first balloon 220. The one or more electrodes 225 can have different shapes, such as circular, oval, band, ring, arcuate, etc. to complete the shape of the balloon. For example, the electrodes 225 can be a series of discrete continuous bands that circumferentially surround the first balloon 220.

In one embodiment, there are a plurality of electrodes 225 that are disposed about the exterior of the first balloon 220. The plurality of electrodes 225 can be arranged in a uniform manner or in a non-uniform manner across the exterior of the first balloon 220. For example, the plurality of electrodes 225 can be arranged in two or more circumferential bands that extend around the first balloon 220. The spacing between the electrodes 225 can be uniform or in other embodiment can be non-uniform in that one or more regions of the first balloon 220 can includes a greater density of the electrodes 225.

### Electrical System

The electrodes 225 are connected to conductive traces 229 that permit an electric pathway to the electrodes 225 from an external stimulation device (generator) shown by reference character 300 in the figures. Any number of different external stimulation devices can be used in the present system 100 so long as they are suitable for the intended application. Electrical stimulation devices (electrical stimulus generator) is configured to generate electrical impulses (electrical stimulus) that stimulates target tissue (the SPG). As is known, the electrical stimulus generator includes circuits that are tailored to deliver a controlled stimulating voltage signal having desired profile. It will also be appreciated that the electrical stimulation device can include one or more selectable stimulation modes. In one embodiment, the electrical stimulation device can be made of disparate transistors and can use an electrically controlled switch that is chosen because of its very low resistance of 4Ω and a charge injection of 110 pC. A low on-resistance increases the accuracy of the stimulation by ensuring that the electrode impedance is not impacted by the switch impedance. The low charge injection is useful because a requirement for safe neural stimulation is charge balancing, which can be ruined by additional charge injected by the switch.

As mentioned, the electrodes 225 are placed on the outside of the balloon and the conductive traces 229 can be routed along or through or parallel to the main catheter and out of the nose of the patient, providing access to the external stimulation device. The ends of the traces can have connectors to allow for easy attachment to the external stimulation device. In the appended figure, the conductive traces 229 are shown routed along the exterior of the main catheter and can be routed through a sleeve 231 that itself is routed along the main catheter. The various conductive traces 229 can be routed through one common sleeve 231. As mentioned herein, the conductive traces can be routed in alternative ways including internally through the main catheter.

As mentioned, one of the major components of the present system is the electrical system 300 which can be considered to include the plurality of the electrodes 225 as well as a master controller 310 that is operatively connected to each electrode 225 to selectively control the supplied energy to the respective electrode(s) 225. The master controller 310 can be a processor that executes software that can be part of a computing device, such as a computing device that is part of a console to which the system 100 is operatively coupled. For example, the console can include a number of controls, connections, and ports. For example, the console can be in fluid communication with or contain a reservoir storing the inflation fluid for inflating each of the two balloons 220, 230. Controls can be provided to instruct independent, controlled inflation of each of the first and second balloons 220, 230, as well as deflation of each of the first and second balloons 220, 230. In addition, the master controller 310 can be configured, in one embodiment, to power on all electrodes 225 at the same time or in another embodiment, the user can select certain electrodes 225, such as certain electrode groups, that can be powered on. Thus, in its simple form, an on/off switch can be provided to activate and then subsequently deactivate the electrodes 225. Based on imaging of the site, selected electrodes 225 can be selected for powering on to apply electrical stimulation to the adjacent tissue that is in contact with one or more of the electrodes 225.

As discussed herein, the console can be a battery powered portable unit to allow for easy transportation and use.

In addition, the controls can be used to control the stimulation intensity of the electrodes 225. There can also be different operating modes for the electrodes 225. For example, the frequency of the stimulation can be varied and/or the stimulation intensity can be varied and selected. In addition, the amplitude of the stimulation is selected and programmed to be an effective dose but less than what would provoke discomfort or have other adverse effects on the patient.

In addition, the electrodes can be stimulated in monopolar, bipolar, or multipolar fashion and be used in a coordinated fashion to steer current to the sphenopalatine ganglion for maximal therapeutic effect.

A display, such as a touch screen, can also be supplied as part of the console to permit display of certain information, such as selected parameters, visualization, etc. In addition, the user interface can be displayed on the display as well.

In yet another embodiment, an external device can be used and can be configured to be temporarily attached to the patient's cheek to allow certain tasks to be performed. For example, these tasks can include actuation (activation) of the electrodes 225 as well as control over the delivery of the inflation fluid to the first balloon 220 and/or second balloon 230. In other words, a small control panel can be temporarily secured to the patient to allows certain selected tasks to be performed. For example, this small control panel can include an on/off switch that operatively controls the operation of the electrodes 225 (i.e., activates and deactivates the electrodes 225).

### Visualization

In yet another aspect, the system 100 can include visualization markers or the like to permit visualization of one or more elements of the system 100 which allows the placement of the system 100 to be confirmed. For example, the electrodes 225 are radiopaque and therefore will serve as markers for placement relative to anatomic landmarks when imaged using traditional imaging systems (techniques) such as x-ray, fluoroscopy, CT, etc. In addition, the first and second balloons 220, 230 can include radiopaque elements, such as radiopaque stripes (bands) or the like as is known.

The inclusion of visualization markers allows for direct visualization of the balloon catheter 200 and thus permits confirmation of its location (relative to SPG).

It will be appreciated that other visualization techniques can be used.

### EXAMPLE

The following example is representative of one exemplary embodiment of the present system; however, it is not to be construed as being limiting of the scope of the present system.

The first balloon 220 can be formed of a polyvinyl chloride (PVC) material; have a major axis/minor axis of 10 mm/7.5 mm; and have a volume of 2.35 ml. In an inflated state, it has a volume of 20 ml in one embodiment. The first balloon 220 can thus be a compliant spherical shaped balloon.

The second balloon 230 can be formed of a polyvinyl chloride (PVC) material; have a major axis/minor axis of 12.5 mm/8 mm; and have a volume of 3.1 ml. In an inflated state, it has a volume of 15 ml in one embodiment.

The main catheter body 210 (catheter shaft) can have an outer diameter of 5.8 mm and an inner diameter of 4 mm. In one embodiment, the first conduit (first tube) 222 and the second conduit (second tube) 224 can have an outer diameter of 1.9 mm and an inner diameter of 1.7 mm.

The guide wire 10 can have a diameter of 2 mm.

### Electrical System

As mentioned, the system 100 includes the use of stimulation energy and in particular, includes one or more stimulating electrodes for providing suitable electrical stimulation (e.g., electrical impulses) to the SPG.

As mentioned, many different types of electrical stimulators can be used to provide electrical stimulation having desired properties to the SPG. For example, the electrical stimulator (system 300) can be constructed using readily available circuit components configured on a customer printed circuit board (PCB) and connected to a power source. For example, a pulse width modulator (PWM) may provide pulses to a circuit. The electrical pulses from the PWM may have the durations or frequencies of their high and low states regulated by a circuit design adapted specifically for this electrical stimulation application. The circuit may further consist of a series of several Bipolar Junction Transistors (BJTs). The BJTs may be configured onto a PCB housing all or part of the circuit. Many of the BJTs in circuit can be supplied through an off-the-shelf component such as a Quad Matched BJT Integrated Circuit (IC) component, for instance a MAT14ARZ. This BJT IC component houses several BJTs connected to pins. Each pin represents a BJT. Pins can be further wired to other components on the PCB.

In one embodiment, circuit can be further wired in such a way that the BJTs operate as current mirrors. A current mirror is a circuit designed to copy a current through one active device by controlling the current in another active device of a circuit, keeping the output current constant regardless of loading. The current being “copied” can be, and sometimes is, a varying signal current.

The electrical stimulator circuit can further comprise additional off-the-shelf semiconductor components, including operational amplifiers (op amps), standalone BJTs components, and electrically controlled switches. In some embodiments, metal-oxide-semiconductor field-effect transistors (MOSFETs) may be used instead of BJTs.

In one embodiment, the op amp may be a LF411CP op amp, because this and similar op amp components work well in a single supply source configuration, such as in circuit. Texas Instruments, Microchip Technologies, and Analog Devices all sell similar semiconductor components which could be used in this type of application. In one embodiment, the standalone BJTs may be a 2N3906-AP component, because it has very similar electrical properties and behavior to the MAT14 transistors found in in the MAT14ARZ IC referenced above.

In some embodiments, the electrically controlled switch may be an ADG621BRMZ component. This type of switch may be desirable due to its low “on-resistance” property and low “charge injection” property. Electrical switches regulate signal transmission into a circuit. “On resistance” is defined as the total measured resistance from the input to output pins of a switch, the switch being is configured in a circuit (or on a PCB). A low on resistance may be desirable in this application because it can increase the accuracy of stimulation by ensuring that electrode impedance is not impacted by switch impedance. “Impedance” is defined as the effective resistance of an electric circuit or component to alternating current, arising from the combined effects of ohmic resistance and reactance. “Charge injection” is typically referred to in the context of parasitic capacitance. Stray capacitance is often associated with transistors or components that make up an analog switch. Minimizing charge injection in this context will enable more consistent charge balancing. “Charge balancing” is in this application involves detecting residual charge by monitoring electrode voltages just before stimulation. If the voltage difference between electrodes is above a certain threshold-implemented through circuit customization and configuration-then a balance current is generated to achieve near net-zero charge at the electrode. Effective charge balancing-and hence a low charge injection-is important in this application because it is a requirement to ensure safe neural stimulation. Such performance features are enabled, at least, by the components listed above, though it is to be understood that the electrical stimulator could be comprised of other or alternative circuit components or configurations which could accomplish a similar electrical stimulation.

However, it will be understood that the preceding discussion of certain suitable components of the electrical stimulator is only exemplary in nature and is not limiting of the scope of the present disclosure since many different types of electrical stimulators having different circuit architecture can be configured and used.

### Method of Treatment (FIGS. 9 and 10)

Upon confirmation of AIS, the balloon catheter 200 is inserted through the nose and into the nasal cavity 1 and the distal end 202 is moved past the end of the septum into the nasopharynx 2. More particularly, the second balloon 230 is designed to travel until it reaches the back of the septum and it completely contacts the nasopharynx 2 once it is fully inflated. As mentioned, the guide wire 10 that extends through the guide wire port 205 can be used to position the balloon catheter 200 within the surgical site at a target location (e.g., into the nasopharynx 2).

Following insertion, inflation fluid, such as saline, can be introduced through the second conduit 224 to deploy the inelastic, elliptical shaped second balloon 230, thereby anchoring the balloon catheter 200 in the nasopharynx 2. The inflation of the second balloon 230 keeps the electrodes stationary and provides accessibility to locate the SPG 3, via geometry, allowing the first balloon 220 to be positioned correctly and the surgeon removes the guide wire 10. The second balloon 230 thus acts as a locking balloon that expands and fills the nasopharynx 2 (the area behind the SPG location) resulting in the balloon catheter 200 being held in place.

In one embodiment, using an external control device (such as one temporarily attached to the patient's cheek or positioned at another location), the spherical shaped first balloon 220 is activated and inflated until the electrodes 225 are fully in contact with the SPG 3 or are located in proximity thereto. The first balloon 220 is thus configured to contact the SPG 3 (or be in close proximity) in the nasal cavity 1 right after the turbinates. In other words, the electrodes 225 can either be in direct contact with the SPG 3 or they can be in non-contact but in close proximity to the SPG at a distance that remains effective for electrical stimulation of the SPG 3 by means of the electrodes 225. Once the first balloon 220 is inflated and is in the proper position, stimulation occurs allowing the system 100 to augment cerebral blood flow (CBF). The first balloon 220 is thus intended to be deployed after the second balloon 230 and it acts to deploy the electrodes 225 (FIG. 8) that are on the first balloon 220.

The treatment can be either acute (over a short period of time, hours or days) or chronic (over an extended period, month to years). In addition, the stimulation regimen selected determines the biological effect: a mild stimulation profile leads to gentle augmentation of cerebral perfusion aiding in the management of ischemic stroke or dementia, while a different, more intense regimen enhances the bioavailability of drugs in the CNS by increasing the permeability of the blood-brain barrier (BBB).

While the present disclosure describes the use of electrodes 225 that are disposed along the balloon, it will be appreciated that other possible arrangements are equally possible so long as they are able to emit electrical stimulation and act like an electrode. For example, the balloon can include one or more regions of a conductive material, such as a coating of a conductive material, that is operatively connected to an electrical trace that itself is placed in operative connection to an electrical stimulation source (stimulator). In the way, electrical stimulation is provided by actuating the regions of the conductive material. The conductive material can be formed in different shapes and patterns, such as a stripe or partial stripe or arcuate segment.

The present system 100 can be described as a “lock and shock” device in that the balloon catheter 200 is first locked in place and then a shocking action occurs by activating the electrodes 225 to delivery electrical stimulation to the target site (e.g., SPG).

The present system 100 also has a number of potential applications and can be used in different settings. The system 100 can be deployed in ambulatory and ER settings. Other settings include but are not limited to a mobile setting during patient transfer. The system 100 is thus designed to improve current treatment strategies.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. A non-invasive neuromodulation device that is configured to stimulate the sphenopalatine ganglion for increasing collateral blood flow to the brain to preserve neural tissue following an acute ischemic stroke, the device comprising:
a catheter having a proximal end and an opposing distal end, the catheter having an inner lumen formed therein;
a first balloon coupled to and surrounding the catheter, the first balloon including at least one electrode; and
a second balloon coupled to and surrounding the catheter;
wherein the first balloon and the second balloon are configured and are spaced apart from one another such that in inflated states of the first balloon and the second balloon, the first balloon is configured to contact or be proximate to the sphenopalatine ganglion to permit stimulation thereof by actuation of the at least one electrode, while the second balloon is configured to hold the non-invasive neuromodulation device in place within the nasopharynx.

2. The device of claim 1, further including a first conduit disposed within the inner lumen and being in fluid communication with the first balloon for inflation thereof and a second conduit disposed within the inner lumen and being in fluid communication with the second balloon for inflation thereof.

3. The device of claim 2, wherein the first conduit and the second conduit are separate and independent from one another to permit independent inflation of the first balloon and the second balloon.

4. The device of claim 2, wherein the first conduit comprises a first tube that extends longitudinally within the inner lumen and the second conduit comprises a second tube that extends longitudinally within the inner lumen.

5. The device of claim 1, further including a handle at a proximal end of the catheter, the handle including first and second inflation fluid ports and a guide wire port, the first inflation fluid port being in fluid communication with an inside of the first balloon via a first conduit and the second inflation fluid port being in fluid communicated with an inside of the second balloon via a second conduit, the guide wire port configured to receive a semi-flexible guide wire.

6. The device of claim 5, wherein the distal end of the catheter is open to permit passage of the semi-flexible guide wire.

7. The device of claim 1, wherein each of the first balloon and the second balloon has a spherical shape, the second balloon having dimensions that are greater than dimensions of the first balloon.

8. The device of claim 1, wherein a distance between the first balloon and the second balloon is between 2 cm and 20 cm.

9. The device of claim 1, wherein the at least one electrode comprises a plurality of electrodes.

10. The device of claim 9, wherein the plurality of electrodes are disposed circumferentially around the first balloon.

11. The device of claim 9, wherein the plurality of electrodes are uniformly distributed across the first balloon.

12. The device of claim 1, wherein the first balloon has dimensions of 10 mm/7.5 mm, as measured along a major axis/minor axis, and the second balloon has dimensions of 12.5 mm/8 mm, as measured along a major axis/minor axis.

13. The device of claim 12, wherein the first balloon has a volume of 2.35 ml and the second balloon has a volume of 3.1 ml.

14. The device of claim 4, wherein the catheter has an outer diameter of 5.8 mm and an inner diameter of 4 mm; the first tube has an outer diameter of 1.9 mm and an inner diameter of 1.7 mm; and the second tube has an outer diameter of 1.9 mm and an inner diameter of 1.7 mm.

15. The device of claim 1, wherein the catheter comprises a flexible tube.

16. The device of claim 1, wherein the second balloon is located distal to the first balloon.

17. The device of claim 1, further including an external actuator that is operatively coupled to the at least one electrode for controlling operation thereof.

18. The device of claim 17, wherein the external actuator is configured to be temporarily attached to a cheek of a patient.

19. The device of claim 17, wherein the external actuator comprise an electrical stimulator.

20. The device of claim 17, wherein the external actuator includes at least two different operating modes in which intensity of the electrical stimulation differs between the modes.

21. The device of claim 1, wherein each of the first and second balloons is filled with inflation media that causes the first and second balloons to be visible during fluoroscopy.

22. The device of claim 21, wherein the inflation media comprises one of saline and a contrast agent that is visible under fluoroscopy.

23. The device of claim 1, wherein the at least one electrode is operatively connected to a conductive trace that is routed either through the inner lumen of the catheter or along an exterior of the catheter.

24. The device of claim 23, wherein the at least one electrode comprises a plurality of electrodes disclosed and spaced apart along an exterior of the first balloon, each electrode having one conductive trace operatively connected thereto, the conductive traces being routed either through a sleeve that extends along an exterior of the catheter or through the inner lumen of the catheter.

25. The device of claim 24, wherein each electrode can be stimulated in monopolar, bipolar or multipolar modes.

26. The device of claim 25, wherein stimulation of the electrodes by a stimulation generator is coordinated to steer current to the sphenopalatine ganglion for optimal therapeutic effect.

27. A method of stimulating the sphenopalatine ganglion for increasing collateral blood flow to the brain to preserve neural tissue following an acute ischemic stroke, the method comprising the steps of:
deploying a non-invasive neuromodulation device intranasally, the non-invasive neuromodulation device including a catheter, a first balloon and a second balloon, the first balloon being coupled to and surrounding the catheter, the first balloon including at least one electrode, the second balloon coupled to and surrounding the catheter;
positioning the non-invasive neuromodulation device such that the first balloon is located proximate the sphenopalatine ganglion and the second balloon is located within the nasopharynx;
inflating the second balloon to retain the non-invasive neuromodulation device within the nasopharynx;
inflating the first balloon resulting in at least the at least one electrode of the first balloon contacting the sphenopalatine ganglion; and
electrically stimulating the sphenopalatine ganglion using the at least one electrode that is disposed along the first balloon.

28. The method of claim 27, further including the step of inserting a guide wire through the catheter to guide the non-invasive neuromodulation device to a target location.

29. The method of claim 27, wherein the step of deploying the non-invasive neuromodulation device intranasally comprises inserting the non-invasive neuromodulation device through the nose and into the nasal cavity.

30. The method of claim 27, wherein the step of inflating the first balloon comprising inflating the first balloon until the at least one electrode is in contact with the sphenopalatine ganglion.

31. The method of claim 27, wherein the first balloon is inflated independent of inflation of the second balloon.

32. The method of claim 27, wherein each of the first balloon and the second balloon has a spherical shape, the second balloon having dimensions that are greater than dimensions of the first balloon.

33. The method of claim 27, wherein a distance between the first balloon and the second balloon is between 2 cm and 20 cm.

34. The method of claim 27, wherein the at least one electrode comprises a plurality of electrodes.

35. The method of claim 34, wherein the plurality of electrodes are disposed circumferentially around the first balloon.

36. The method of claim 27, wherein the first balloon has dimensions of 10 mm/7.5 mm, as measured along a major axis/minor axis, and the second balloon has dimensions of 12.5 mm/8 mm, as measured along a major axis/minor axis.

37. The method of claim 36, wherein the first balloon has a volume of 2.35 ml and the second balloon has a volume of 3.1 ml.

38. The method of claim 27, wherein the second balloon is located distal to the first balloon.

39. The method of claim 27, further including the step of using an external actuator that is operatively connected to the at least one electrode for controlling operation thereof to provide the stimulation.

40. The method of claim 39, wherein the external actuator is configured to be temporarily attached to a cheek of a patient.

41. The method of claim 39, wherein the external actuator comprises an electrical stimulator.

42. The method of claim 39, wherein the external actuator includes at least two different operating modes in which intensity of the electrical stimulation differs.

43. The method of claim 27, wherein the step of deploying the non-invasive neuromodulation device intranasally comprises bending the catheter to position the first balloon proximate the sphenopalatine ganglion and the second balloon within the nasopharynx.

44. A method of stimulating the sphenopalatine ganglion for increasing collateral blood flow to the brain to preserve neural tissue following an acute ischemic stroke, the method comprising the steps of:
deploying a non-invasive neuromodulation device intranasally; and
electrically stimulating the sphenopalatine ganglion with at least one electrode.

45. The method of claim 44, wherein the non-invasive neuromodulation device comprises a balloon catheter including a first balloon and a second balloon, the first balloon including the least one electrode, the second balloon being located distal to the first balloon along a shaft of the balloon catheter.

46. The method of claim 45, wherein the non-invasive neuromodulation device is positioned such that the first balloon is located adjacent the sphenopalatine ganglion and the second balloon is located within the nasopharynx.

47. The method of claim 46, wherein the second balloon is inflated prior to inflation of the first balloon to securely hold the non-invasive neuromodulation device within the nasopharynx prior to inflating the first balloon to contact the at least one electrode with the sphenopalatine ganglion.

48. The method of claim 44, wherein the step of electrically stimulating the sphenopalatine ganglion with the at least one electrode comprises using an external actuator to control delivery of the electrical stimulation.

49. The method of claim 48, wherein the at least one electrode is radiopaque and the method further includes the step of using an image system for determining a location of the non-invasive neuromodulation device relative to anatomic landmarks as a result of the at least one radiopaque electrode.

50. The method of claim 49, wherein the image system comprises one of an x-ray device, a fluoroscopy and a CT device.

51. The method of claim 45, further including the step of: inflating the first and second balloons using an inflation media that causes the first and second balloons to be visible during fluoroscopy.

52. The method of claim 51, wherein the inflation media comprises one of saline and a contrast agent that is visible under fluoroscopy.

53. The method of claim 45, wherein the at least one electrode is operatively connected to a conductive trace that is routed either through an inner lumen of the shaft of the catheter or along an exterior of the shaft of the catheter.

54. The method of claim 53, wherein the at least one electrode comprises a plurality of electrodes disclosed and spaced apart along an exterior of the first balloon, each electrode having one conductive trace operatively connected thereto, the conductive traces being routed either through a sleeve that extends along an exterior of the shaft of the catheter or through the inner lumen of the shaft of the catheter.

55. The method of claim 54, wherein each electrode can be stimulated in monopolar, bipolar or multipolar modes.

56. The method of claim 55, wherein stimulation of the electrodes by a stimulation generator is coordinated to steer current to the sphenopalatine ganglion for optimal therapeutic effect.
